(19) United States
(12) Patent Application Publication
(10) Pub. No.: US 2022/0193021 A1
(43) Pub. Date: Jun. 23, 2022

(54) COMBINATION THERAPY FOR TREATING INFLUENZA VIRUS INFECTION

(71) Applicant: President and Fellows of Harvard College, Cambridge, MA (US)

(72) Inventors: Longlong Si, Cambridge, MA (US); Rachelle Prantil-Baun, Cambridge, MA (US); Kambez Hajipouran Benam, Cambridge, MA (US); Melissa Rodas, Cambridge, MA (US); Ratnakar Potla, Cambridge, MA (US); Donald E. Ingber, Cambridge, MA (US)

(73) Assignee: President and Fellows of Harvard College, Cambridge, MA (US)

(21) Appl. No.: 17/608,452

(22) PCT Filed: Apr. 30, 2020
(86) PCT No.: PCT/US2020/030697

§ 371 Date: Nov. 2, 2021

Related U.S. Application Data
(60) Provisional application No. 62/842,803, filed on May 3, 2019.

(51) Int. Cl. A61K 31/245; A61P 31/16

(52) U.S. Cl. CPC A61K 31/245; A61P 31/16

(57) ABSTRACT

Provided herein, in some aspects, are combination therapies for inhibiting influenza virus infection.

### RELATED APPLICATION

This application claims the benefit under 35 U.S.C. § 119(e) of U.S. provisional application No. 62/842,803, filed May 3, 2019, which is incorporated by reference herein in its entirety.

### GOVERNMENT LICENSE RIGHTS

This invention was made with government support under HL141797 awarded by National Institutes of Health. The government has certain rights in the invention.

### BACKGROUND

Influenza is a disease caused by influenza virus infection of the respiratory tract epithelium that has global impact causing enormous morbidity and mortality every year. All of current anti-influenza drugs approved for clinical use have been designed to specifically target viral proteins, however, the high mutation rate of viral proteins has led to the emergence of drug-resistant influenza viruses in clinical settings, limiting the use of those drugs. Further, most patients with influenza do not become aware they are infected by virus until clinical symptoms develop, and this is days after the initial infection and window during which currently-approved anti-influenza drugs are effective.

### SUMMARY

Methods and compositions of the present disclosure are based on unexpected data demonstrating a synergistic effect on the reduction of influenza virus titers following administration of the combination of nafamostat and oseltamivir to lung airway cells infected with the virus. Further, while oseltamivir alone provides limited protection in patients when treated two days (48 hours) post infection, the data herein suggests that co-administration of oseltamivir with nafamostat mesylate (also known as NAFAMOSTAT®, nafamostat mesilate, nafamostat mesylate, FUT-175, nafamstat, nafamostat dihydrochloride, nafamostat hydrochloride, nafamostat HCl, and ronastat) will extend the therapeutic window of treatment for influenza virus infection through at least four days (96 hours) post infection.

In some aspects, the present disclosure is a method for reducing influenza virus titer in a subject, comprising administering to a subject infected with influenza virus a combination of nafamostat mesylate and oseltamivir in an effective amount to reduce influenza virus titer in the subject by at least 20% relative to a control. The control may be a subject infected with influenza that is not administered a combination of nafamostat mesylate and oseltamivir, a subject that is administered an effective amount of nafamostat mesylate, or a subject that is administered an effective amount of oseltamivir.

In some embodiments, the combination is administered to the subject within 24 hours of infection in an effective amount to reduce influenza virus titer in the subject by at least 20% relative to a control. In some embodiments, the control is a subject administered only nafamostat mesylate or only oseltamivir within 24 hours of influenza virus infection.

In some embodiments, the combination is administered to the subject within 48 hours of infection in an effective amount to reduce influenza virus titer in the subject by at least 20% relative to a control. In some embodiments, the control is a subject administered only nafamostat mesylate or only oseltamivir within 48 hours of influenza virus infection.

In some embodiments, the combination is administered to the subject within 72 hours of infection in an effective amount to reduce influenza virus titer in the subject by at least 50% relative to a control. In some embodiments, the control is a subject administered only nafamostat mesylate or only oseltamivir within 72 hours of influenza virus infection.

In some embodiments, the combination is administered to the subject within 96 hours of infection in an effective amount to reduce influenza virus titer in the subject by at least 20% relative to a control. In some embodiments, the control is a subject administered only nafamostat mesylate or only oseltamivir within 96 hours of influenza virus infection.

In some embodiments, the nafamostat mesylate and the oseltamivir may be administered simultaneously. In any of the embodiments described herein, the nafamostat mesylate and oseltamivir may be administered sequentially. In some embodiments where the nafamostat mesylate and oseltamivir are administered sequentially, the nafamostat mesylate is administered before the oseltamivir.

In some embodiments, the nafamostat mesylate is administered in an effective amount to prevent emergence of influenza virus strains that are resistant to nafamostat mesylate. In some embodiments, the nafamostat mesylate is administered in an effective amount to reduce emergence of influenza virus strains that are resistant to nafamostat mesylate by at least 60% relative to a control.

In some aspects, the present disclosure is a pharmaceutical composition comprising nafamostat mesylate and oseltamivir formulated in effective amounts to prevent and/or treat influenza virus infection in a subject.

In some aspects, the present disclosure is a method for reducing influenza virus resistance in a subject, comprising administering to a subject infected with influenza virus an effective amount of nafamostat mesylate, or a combination of nafamostat mesylate and oseltamivir, in an effective amount to reduce influenza virus resistance in the subject by at least 60% relative to a control. The control may be a subject infected with influenza virus that is administered an effective amount of nafamostat mesylate, a subject that is administered an effective amount of oseltamivir, a subject that is administered an effective amount of another anti-influenza drug (e.g., amantadine), or a subject that is not administered an anti-influenza drug.

### BRIEF DESCRIPTION OF THE DRAWINGS

FIG. 1 shows the efficacy of a drug combination of nafamostat mesylate (10 μM) and oseltamivir (1 μM) against influenza virus. Human lung airway chips were treated with nafamostat mesylate through the air channel and oseltamivir through the blood channel at −24, 0, 24, 48, 72, and 96 hours (h) post-infection. The progeny viruses were collected and quantified by the plaque formation assay.

FIG. 2 shows the activity of nafamostat mesylate (10 μM) against influenza virus strains H1N1 and H3N2. Influenza virus-infected human airway (MOI=0.1) chips were treated with nafamostat mesylate through the air channel. The progeny viruses were collected and quantified by the plaque formation assay.

FIG. 3 shows influenza virus titer in cells expressing serine proteases. Virus replication is increased in cells expressed the transmembrane serine protease 4 (TMPRSS4), transmembrane serine protease 11D (TMPRSS11D), transmembrane serine protease 2 (TMPRSS2), and transmembrane serine protease 11E (DESC1) relative to cells not expressing any of these serine proteases (empty vector).

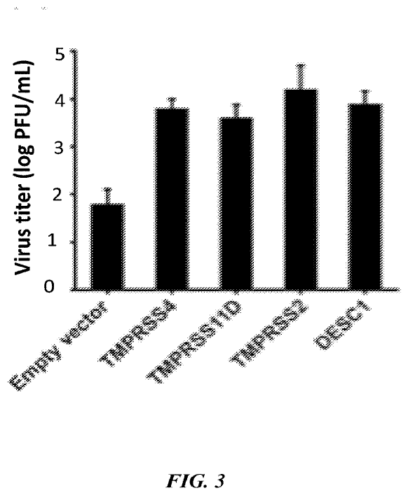

FIG. 4 shows the inhibition of nafamostat mesylate on the cleavage of influenza virus protein hemagglutinin 0 (HA0) into hemagglutinin 1 (HA1) and hemagglutinin 2 (HA2) subunits. Influenza virus-infected human lung airway chip was treated with nafamostat mesylate (10 μM) or vehicle, and the total protein was extracted 48 hours later and subjected to Western blotting of HA0 and HA1.

FIG. 5 shows the relative transcription levels of serine proteases in the presence or absence of nafamostat mesylate. Human lung airway chips were treated with nafamostat mesylate (10 μM) or vehicle, and the mRNA levels of the serine proteases (TMPRSS4, TMPRSS11D, TMPRSS2, and DESC1) were measured. mRNA expression of chips treated with nafamostat mesylate is relative to chips not treated with nafamostat mesylate.

FIG. 6 shows the relative protein levels of serine proteases in the presence or absence of nafamostat mesylate. Human lung airway chips were treated with nafamostat mesylate (10 μM) or vehicle, and the protein levels of the serine proteases (TMPRSS4, TMPRSS11D, TMPRSS2, and DESC1) were measured. Protein levels of chips treated with nafamostat mesylate are relative to chips not treated with nafamostat mesylate.

FIGS. 7A-7B show influenza virus resistance to nafamostat mesylate. FIG. 7A shows influenza viral plaques of progeny virus at the 1^{st} passage (P1) and the 30^{th} passage (P30). FIG. 7B shows influenza viral plaque titers at P1 and P30. Naf is nafamostat mesylate, “+” is nafamostat-treated human lung airway chips, “−” is human lung airway chips not treated with nafamostat, and DMSO is dimethyl sulfoxide.

FIGS. 8A-8B show influenza virus resistance to oseltamivir. FIG. 8A shows influenza viral plaques of progeny virus at the 1^{st} passage (P1) and the 25^{th} passage (P25). FIG. 8B shows influenza viral plaque titers at P1 and P25. Osv is oseltamivir, “+” is oseltamivir-treated human lung airway chips, “−” is human lung airway chips not treated with oseltamivir, and DMSO is dimethyl sulfoxide.

FIGS. 9A-9B show influenza virus resistance to amantadine. FIG. 9A shows influenza viral plaques of progeny viruses at the 1^{st} passage (P1) and the 8^{th} passage (P8). FIG. 9B shows influenza viral plaque titers at P1 and P8. “+” is amantadine-treated human lung airway chips, “−” is human lung airway chips not treated with amantadine, and DMSO is dimethyl sulfoxide.

### DETAILED DESCRIPTION

The present disclosure provide methods and compositions for reducing influenza virus titer in a subject with a combination of nafamostat mesylate and oseltamivir. As shown herein, the combination of nafamostat mesylate and oseltamivir is synergistic and extends the therapeutic window of influenza treatment by more than 24 hours compared with either nafamostat mesylate or oseltamivir individually. Thus, the combination of nafamostat mesylate and oseltamivir is more effective at reducing influenza virus titer than either nafamostat mesylate or oseltamivir individually.

The influenza virus replication inhibition and inhibition of release of influenza virus from infected cells of nafamostat mesylate and oseltamivir was investigated utilizing a human lung airway chip device. Thus, the present disclosure also provides a human lung airway chip device that may be used, for example, for identifying additional combination influenza inhibition therapies.

In some aspects, the present disclosure provides methods of contacting an airway cell with nafamostat mesylate and oseltamivir. The human airway is composed of the pharynx, larynx, trachea, bronchi, bronchioles, alveoli and is lined with epithelial and endothelial cells. In some embodiments, the airway cells are lung airway epithelial cells (e.g., human lung airway epithelial cells). In some embodiments, the airway cells are lung microvascular endothelial cells (e.g., human lung microvascular endothelial cells). The cells, in some embodiments, are epithelial cells, endothelial cells, or a combination of epithelial cells and endothelial cells.

Nafamostat mesylate (Nafamostat®) an FDA approved anticoagulant that inhibits serine protease inhibitors that convert fibrinogen into fibrin in blood clotting. Serine protease expression is elevated in human airway cells, and serine protease activity is required for the cleavage of the inactive influenza hemagglutinin-0 (HA0) protein into active hemagglutinin-1 (HA1) and hemagglutinin-2 (HA2) proteins. Cleavage and activation of HA0 into HA1 and HA2 proteins is required for entry of the influenza virus into airway cells (e.g., epithelial cells, endothelial cells). Thus, contacting an airway cell that is infected with influenza with nafamostat mesylate may decrease the influenza virus replication by preventing activation of the influenza virus.

Oseltamivir (TAMIFLU®) is an FDA approved anti-influenza drug that inhibits the neuraminidase enzyme. Neuraminidase is an enzyme expressed on the surface of an influenza virus particle that promotes the release of influenza virus from an infected cell and facilitates viral movement throughout the airway. In the presence of oseltamivir, influenza virus particles remain attached to the membrane of infected cells and trapped in airway secretions (e.g., mucus). Thus, contacting an airway cell that is infected with influenza with oseltamivir may decrease influenza virus release from infected cells.

Influenza virus infects hosts such as humans and livestock animals (e.g., cattle, sheep, goats, poultry, or pigs). Infection can result in global pandemic as the virus spreads among hosts who are contagious but have not yet developed symptoms of infection. Influenza virus primarily infects cells of the airway (e.g., lung epithelial, airway epithelial, and/or alveoli) before spreading throughout the body. The symptoms of influenza virus infection include, for example, congestion, cough, sore throat, fever, chills, aches, and fatigue, and typically appear two days after exposure to the virus and last for less than a week. In more severe cases, complications of influenza virus infection can lead to pneumonia, secondary bacterial pneumonia, sinus infection, and worsening of previous health problems, including asthma or heart failure. In the most severe cases, influenza virus infection can lead to death, particularly in young children, the elderly, and immunosuppressed subjects.

Oseltamivir inhibits the influenza enzyme neuraminidase. Although oseltamivir is initially effective as an anti-influenza drug, resistance to oseltamivir can develop in influenza-infected cells. In some aspects, the present disclosure provides methods of contacting an airway cell with nafamostat mesylate and oseltamivir in effective amounts to reduce influenza virus titer. An effective amount of nafamostat mesylate and/or oseltamivir used in the methods provided herein may vary and may be determined by a skilled practitioner. The effective amount of nafamostat mesylate and/or oseltamivir may depend at least in part on the stage of viral infection, the severity of infection, and/or the sex, age, height, weight, and/or general health of the subject.

In some embodiments, the effective amount of nafamostat mesylate inhibits replication of the influenza virus. Replication of a virus can be determined/monitored by measuring viral titer, for example. Viral titer is a measure of the quantity of virus in a given volume. Non-limiting methods of measuring viral titer include viral plaque assay (Baer and Kehn-Hall, 2014, J Vis Exp. 93: 52056; Yu, et al., 2014, J Med Chem, 57, 10058-10071), quantitative polymerase chain reaction (qPCR) of viral proteins (Zhang and Evans, 1991, J. Virol Methods, 33: 165-189; Vester, et al., 2010, J. Virol Methods, 168: 63-71, Si, et al., 2016 Science 354: 1170-1173), 50% tissue culture infectious dose assay (TCID50) (LaBarre and Lowy, 2001, J. Virol Methods, 96: 107-126), and focus forming assay (Nutter, et al., 2012, PLoS One, 7: e33097). A decreased viral titer relative to a subject who has not been treated with nafamostat mesylate is indicative of a decrease in viral replication and thus viral spread. An increased viral titer relative to a subject who has not been treated with nafamostat mesylate is indicative of an increase in viral replication and thus viral spread.

In some aspects, a combination of nafamostat mesylate and oseltamivir is administered in an effective amount to reduce influenza virus titer in the subject by at least 20% (e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%) relative to a control. In some aspects, a combination of nafamostat mesylate and oseltamivir is administered in an effective amount to reduce influenza virus titer in the subject by 20%-100%, 30%-100%, 40%-100%, 50%-100%, 20%-90%, 30%-90%, 40%-90%, 50%-90%, 20%-80%, 30%-80%, 40%-80%, or 50%-80%, relative to a control. The control may be a subject infected with influenza that is not administered a combination of nafamostat mesylate and oseltamivir, a subject that is administered an effective amount of nafamostat mesylate, or a subject that is administered an effective amount of oseltamivir.

In some embodiments, the combination is administered to the subject within 96 hours (e.g., within 72 hours, within 60 hours, within 48 hours, within 32 hours, within 24 hours, within 18 hours, within 12 hours, or within 6 hours) of infection in an effective amount to reduce influenza virus titer in the subject by at least 20% (e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%) relative to a control. In some embodiments, the combination is administered to the subject within 96 hours (e.g., within 72 hours, within 60 hours, within 48 hours, within 32 hours, within 24 hours, within 18 hours, within 12 hours, or within 6 hours) of infection in an effective amount to reduce influenza virus titer in the subject by 20%-100%, 30%-100%, 40%-100%, 50%-100%, 20%-90%, 30%-90%, 40%-90%, 50%-90%, 20%-80%, 30%-80%, 40%-80%, or 50%-80%, relative to a control. In some embodiments, the control is a subject administered only nafamostat mesylate or only oseltamivir within 24 hours of influenza virus infection.

In some embodiments, the combination is administered to the subject within 96 hours (e.g., within 72 hours, within 60 hours, within 48 hours, within 32 hours, within 24 hours, within 18 hours, within 12 hours, or within 6 hours) of infection in an effective amount to reduce influenza virus titer in the subject by at least 1.5-fold (e.g., at least 2-fold, at least 2.5-fold, at least 3-fold, at least 3.5-fold, at least 4-fold, at least 4.5-fold, or at least 5-fold) relative to a control. In some embodiments, the combination is administered to the subject within 96 hours (e.g., within 72 hours, within 60 hours, within 48 hours, within 32 hours, within 24 hours, within 18 hours, within 12 hours, or within 6 hours) of infection in an effective amount to reduce influenza virus titer in the subject by 1.5-fold to 5-fold, 2-fold to 5-fold, 2.5-fold to 5-fold, or 3-fold to 5-fold, relative to a control. In some embodiments, the control is a subject administered only nafamostat mesylate or only oseltamivir within 24 hours of influenza virus infection.

In some embodiments, the effective amount of nafamostat mesylate is 0.1 μM-100 μM at the drug delivery sites. In some embodiments, the effective amount of nafamostat mesylate is 10 μM-80 μM. In some embodiments, the effective amount of nafamostat mesylate is 5 μM-50 μM. In some embodiments, the effective amount of nafamostat mesylate is 1 μM-20 μM. In some embodiments, effective amount of nafamostat mesylate is 25 μM-75 μM. In some embodiments, the effective amount of nafamostat mesylate is 0.1 μM, 1.0 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM.

Drug delivery sites are regions in or on the subject at which an anti-influenza drug (e.g., nafamostat mesylate and/or oseltamivir) is administered. Non-limiting examples of drug delivery sites are muscles, veins, arteries, the nasal cavity, the oral cavity, the stomach, the liver, the small intestine, and the large intestine.

In some embodiments, the effective amount of oseltamivir inhibits the release of virus from an infected cell. Release of virus can be determined/monitored by measuring viral titer, for example. Viral titer can be measured by any of the methods described above.

In some embodiments, the effective amount of oseltamivir is 10 mg-1000 mg. In some embodiments, the effective amount of oseltamivir is 75 mg-500 mg. In some embodiments, the effective amount of oseltamivir is 150 mg-1000 mg. In some embodiments, the effective amount of oseltamivir is 200 mg-800 mg. In some embodiments, the effective amount of oseltamivir is 400 mg-600 mg. In some embodiments, the effective amount of oseltamivir is 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg.

In some embodiments, when the subject is less than 1 year in age, the effective amount of oseltamivir is 1 mg/kg-30 mg/kg, 3 mg/kg-24 mg/kg, 6 mg/kg-18 mg/kg, 2 mg/kg-28 mg/kg, or 5 mg/kg-25 mg/kg. In some embodiments, when the subject is less than 1 year in age, the effective amount of oseltamivir is 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, or 30 mg/kg.

Replication of the influenza virus and release of influenza virus from infected cells can be detected by measuring viral titer (Baer and Kehn-Hall, 2014, J Vis Exp. 93: 52056; LaBarre and Lowy, 2001, J. Virol Methods, 96: 107-126; Nutter, et al., 2012, PLoS One, 7: e33097), by staining for influenza virus particle proteins in a sample (e.g., neuraminidase, hemagglutinin, nucleoprotein) (Daisy, et al., 1979, J. Clin Microbiol, 9: 688-692; Johnson, et al., 2012, J. Clin Microbiol, 50: 396-400; Xiao, et al., 2016, Biomaterials, 78: 74-85; Si, et al., 2018, Sci Adv, 4, eaau8408), and by measuring the expression of influenza virus proteins in a sample by qPCR (Zhang and Evans, 1991, J. Virol Methods, 33: 165-189; Vester, et al., 2010, J. Virol Methods, 168: 63-71).

In some embodiments, the effective amount of nafamostat mesylate is 0.1 μM-100 μM at the drug delivery sites and the effective amount of oseltamivir is 10 mg-1000 mg in a combination therapy. In some embodiments, the effective amount of nafamostat mesylate is 10 μM-80 μM and the effective amount of oseltamivir is 75 mg-500 mg in a combination therapy. In some embodiments, the effective amount of nafamostat mesylate is 5 μM-50 μM and the effective amount of oseltamivir is 150 mg-1000 mg in a combination therapy. In some embodiments, the effective amount of nafamostat mesylate is 1 μM-20 μM and the effective amount of oseltamivir is 200 mg-800 mg in a combination therapy. In some embodiments, the effective amount of nafamostat mesylate is 25 μM-75 μM and the effective amount of oseltamivir is 400 mg-600 mg in a combination therapy. A combination therapy is administering nafamostat mesylate and oseltamivir to a subject in need thereof.

In some embodiments, the effective amount of nafamostat mesylate is 0.1 μM-100 μM at the drug delivery sites and the effective amount of oseltamivir is 1 mg/kg-30 mg/kg in a combination therapy. In some embodiments, the effective amount of nafamostat mesylate is 10 μM-80 μM and the effective amount of oseltamivir is 3 mg/kg-24 mg/kg in a combination therapy. In some embodiments, the effective amount of nafamostat mesylate is 5 μM-50 μM and the effective amount of oseltamivir is 6 mg/kg-18 mg/kg in a combination therapy. In some embodiments, the effective amount of nafamostat mesylate is 1 μM-20 μM and the effective amount of oseltamivir is 2 mg/kg-28 mg/kg in a combination therapy. In some embodiments, the effective amount of nafamostat mesylate is 25 μM-75 μM and the effective amount of oseltamivir is 5 mg/kg-25 mg/kg in a combination therapy. A combination therapy is administering nafamostat mesylate and oseltamivir to a subject in need thereof.

In some embodiments, replication of influenza virus and release of influenza virus from infected cells is detected by measuring viral titer. Viral titer can be measured by any of the methods described herein. In some embodiments, the effective amounts of nafamostat mesylate and oseltamivir reduces influenza virus titers by 2-fold to 200-fold, 10-fold to 100-fold, 25-fold to 200-fold, 50-fold to 200-fold, 2-fold to 50-fold, 5-fold to 100-fold, or 10-fold to 50-fold relative to a control. A control may be a subject infected with influenza that has not been administered effective amounts of nafamostat mesylate and oseltamivir, the same subject before effective amounts of nafamostat mesylate and oseltamivir are administered, a subject infected with influenza that is administered an effective amount of nafamostat mesylate, or a subject infected with influenza that is administered an effective amount of oseltamivir.

Oseltamivir and nafamostat mesylate reduce influenza viral titers when they are administered to a subject within 48 hours of influenza infection. Unfortunately, not all subjects display clinical symptoms of influenza virus infection within 48 hours. For these subjects, the therapeutic window of administering nafamostat mesylate or oseltamivir is already passed by the time that clinical symptoms appear and they realize they are infected. The therapeutic window of a drug (e.g., nafamostat mesylate, oseltamivir) is the range of drug. Some aspects of this disclosure are based on the unexpected result that administration of both nafamostat mesylate and oseltamivir to a subject infected with influenza extends the therapeutic window to up to 96 hours after influenza infection.

In some embodiments, nafamostat mesylate and oseltamivir are administered to subjects within 24 hours-96 hours after influenza virus infection. In some embodiments, nafamostat mesylate and oseltamivir are administered within 48-72 hours after influenza virus infection. In some embodiments, nafamostat mesylate and oseltamivir are administered within 72 hours after influenza virus infection. In some embodiments, nafamostat mesylate and oseltamivir are administered within 96 hours after influenza virus infection.

Contacting airway cells with nafamostat mesylate and oseltamivir prior to influenza virus may help prevent influenza virus infection by inhibiting influenza virus entry into host cells and/or inhibiting influenza virus release from infected cells. In some embodiments, nafamostat mesylate and oseltamivir contacted with airway cells or administered to subjects prior to influenza virus infection.

Contacting airway cells with nafamostat mesylate, in some embodiments, reduces influenza virus resistance. Influenza virus resistance results in reduced efficacy of influenza virus drugs as treatment progresses. Influenza virus resistance can be measured by multiple methods, including, but not limited to, the viral plaque assay, quantitative polymerase chain reaction (qPCR) of viral proteins, the 50% tissue culture infectious dose assay (TCID50), and the focus forming assay. In some embodiments, administration of nafamostat mesylate reduces influenza virus resistance by at least 60% (e.g., 60%, 65%, 70%, 75%, 80%, 85%, 90%, 95%, 99%, or 100%) relative to a control. A control may be airway cells infected with influenza virus not administered nafamostat or airway cells infected with influenza virus administered another anti-influenza drug (e.g., oseltamivir, amantadine).

Contacting airway cells with a combination of nafamostat mesylate and oseltamivir may reduce influenza virus resistance. In some embodiments, administration of the combination of nafamostat mesylate and oseltamivir reduces influenza virus resistance by at least 60% (e.g., 60%, 65%, 70%, 75%, 80%, 85%, 90%, 95%, 99%, or 100%) relative to a control. A control may be airway cells infected with influenza virus administered oseltamivir, airway cells infected with influenza virus administered nafamostat mesylate, airway cells infected with influenza virus administered another anti-influenza drug (e.g., amantadine), and airway cells infected with influenza virus not administered an anti-influenza drug.

Nafamostat mesylate and oseltamivir may be administered simultaneously or sequentially. In some embodiments, nafamostat mesylate and oseltamivir are administered simultaneously. Simultaneously means that the nafamostat mesylate and the oseltamivir are administered to the subject at the same time. The nafamostat mesylate and oseltamivir may be co-formulated for simultaneous administration (e.g., in the same composition) or formulated separately and administered simultaneously.

The nafamostat mesylate and oseltamivir may also be administered sequentially. In sequential administration, there may be 1 second-1 minute, 1 minute-10 minutes, 5 minutes 30 minutes, 15 minutes-60 minutes (1 hour), 30 minutes-3 hours, 1 hour-6 hours, 2 hours-8 hours, 4 hours-12 hours, 6 hours-18 hours, 12 hours-24 hours (1 day), or 16 hours-1 day between the first administration and the second administration. In some embodiments, nafamostat mesylate is administered before oseltamivir. In some embodiments, oseltamivir is administered before nafamostat mesylate.

Nafamostat mesylate and oseltamivir may be co-formulated for simultaneous administration. The co-formulation may be a composition comprising nafamostat mesylate, oseltamivir, and a pharmaceutically acceptable buffer. A pharmaceutically acceptable buffer is a solution that contains the nafamostat mesylate and oseltamivir that does not produce an adverse effect when administered to the subject. Non-limiting examples of pharmaceutically acceptable buffer include phosphate buffered saline (PBS), tris buffered saline (TBS), 4-(2-hydroxyehtyl)-1-piperazineethanesulfonic acid (HEPES), or hydrochloric acid, tris-acetate-EDTA buffer (TAE), tris-boron-EDTA buffer (TBE), phosphate buffer, ammonium sulfate buffer, maleic acid, tartaric acid, lactic acid, citric acid, acetic acid, sodium bicarbonate, and sodium phosphate. Other buffers may also be used.

In some embodiments, the composition comprises one or more pharmaceutically acceptable carriers. A pharmaceutically acceptable carrier is any substrate used in the process of composition administration to a subject that serves to improve the selectivity, effectiveness, and/or safety of composition administration and does not cause an adverse effect when administered to a subject. Non-limiting examples of pharmaceutically acceptable carriers that may be included in the compositions described herein include liposomes, polymeric micelles, microspheres, nanostructures, nanofibers, protein-DNA complexes, protein-drug conjugates, erythrocytes, virosomes, and dendrimers.

In some embodiments, the composition comprises one or more pharmaceutically acceptable excipients. A pharmaceutically acceptable excipient is a substance included in a composition for improving long-term stabilization of the composition, to confer enhanced influenza virus titer reduction in the subject compared to compositions not comprising the pharmaceutically acceptable excipient, or to aid in the manufacturing of the composition. Non-limiting examples of pharmaceutically acceptable excipients that may be included in the compositions described herein include vehicles (e.g., petrolatum, dimethyl sulfoxide, mineral oil), anti-adherents, binders, coatings, colors, disintegrants, flavors, glidants, lubricants, preservatives, sorbents, and sweeteners.

The composition may be formulated as a solution (e.g., liquid), a suspension (e.g., colloid), a pill, or lyophilized mixture. The composition may be administered by any route known in the art. Non-limiting routes of administration include injection (e.g., intravenous, intramuscular, intraarterial), inhalation, ingestion, topical, and rectal.

The compositions described herein may be formulated as a dosage form for administration to the subject. A dosage form is a pharmaceutical drug product in the form in which it is marketed for use, with the specific mixture of effective amounts of nafamostat mesylate and oseltamivir, pharmaceutically acceptable buffers, pharmaceutically acceptable carriers, and/or pharmaceutically acceptable excipients in a particular configuration. Non-limiting examples of dosage forms include oral (e.g., pill, thin film, liquid solution or suspension, powder, paste), inhalational (e.g., aerosol, inhaler, nebulizer, smoking, vaporizer), parenteral (intradermal, subcutaneous, intramuscular, intraosseous, intraperitoneal, intravenous), topical (cream, gel, liniment, balm, lotion, ointment, ear drops, eye drops, skin patch, powder talc), and suppository (vaginal, rectal urethral, nasal)

The compositions described herein are administered to subjects having an influenza virus infection or suspected of being at risk of developing an influenza virus infection. In some embodiments, the subjects are humans. Human subjects may be any humans that are 2 weeks of age or older. In some embodiments, human subjects are 65 years or older. In some embodiments, human subjects are 1 year or older. In some embodiments, human subjects are immunosuppressed.

In some embodiments, the subjects are livestock animals. Non-limiting examples of livestock animals include pigs, cattle, poultry, sheep, horses, donkeys, and goats. In some embodiments, the subjects are non-human primates. Non-limiting examples of non-human primates include Rhesus monkeys, chimpanzees, orangutans, macaques, and gorillas.

Nafamostat mesylate and oseltamivir are present in the composition in effective amounts for reducing influenza virus titers. In some embodiments, the effective amounts of nafamostat mesylate and oseltamivir are the same. In some embodiments, the ratio of nafamostat mesylate to oseltamivir is 1:1-20:1, 2:1-4:1, 6:1-12:1, 3:1-18:1, or 5:1-20:1. In some embodiments the ratio of nafamostat mesylate to oseltamivir is 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some embodiments, the ratio of nafamostat mesylate to oseltamivir is 1:1-1:20, 1:2-1:4, 1:6-1:12, 1:3-1:18, or 1:5-1:20. In some embodiments, the ratio of nafamostat mesylate to oseltamivir is 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

### Additional Embodiments

Additional embodiments of the present disclosure are provided in the numbered paragraphs below.

1. A method for reducing influenza virus titer in a subject, comprising administering to a subject infected with influenza virus a combination of nafamostat mesylate and oseltamivir in an effective amount to reduce influenza virus titer in the subject by at least 20% relative to a control.

2. The method of paragraph 1, wherein the combination is administered to the subject within 24 hours of infection in an effective amount to reduce influenza virus titer in the subject by at least 20% relative to a control, wherein the control is a subject administered only nafamostat mesylate or only oseltamivir within 24 hours of influenza virus infection.

3. The method of paragraph 1 or 2, wherein the combination is administered to the subject within 48 hours of infection in an effective amount to reduce influenza virus titer in the subject by at least 20% relative to a control, wherein the control is a subject administered only nafamostat mesylate or only oseltamivir within 48 hours of influenza virus infection.

4. The method of any one of the preceding numbered paragraphs, wherein the combination is administered to the subject within 72 hours of infection in an effective amount to reduce influenza virus titer in the subject by at least 50% relative to a control, wherein the control is a subject administered only nafamostat mesylate or only oseltamivir within 72 hours of influenza virus infection.

5. The method of any one of the preceding numbered paragraphs, wherein the combination is administered to the subject within 96 hours of infection in an effective amount to reduce influenza virus titer in the subject by at least 20% relative to a control, wherein the control is a subject administered only nafamostat mesylate or only oseltamivir within 72 hours of influenza virus infection.

6. The method of any one of the preceding numbered paragraphs, wherein the nafamostat mesylate and the oseltamivir are administered simultaneously.

7. The method of any one of the preceding numbered paragraphs, wherein the nafamostat mesylate and oseltamivir are administered sequentially.

8. The method of paragraph 7, wherein the nafamostat mesylate is administered before the oseltamivir is administered.

9. A method for preventing influenza virus infection in a subject, comprising administering to a subject a combination of nafamostat mesylate and oseltamivir in an effective amount to prevent influenza virus infection in the subject.

10. A method comprising contacting an airway cell with nafamostat mesylate and oseltamivir.

11. The method of paragraph 10, wherein the airway cell is contacted with the nafamostat mesylate and oseltamivir in effective amounts for inhibiting influenza virus replication.

12. The method of paragraph 10 or 11, wherein the airway cell is contacted with the nafamostat mesylate and oseltamivir in effective amounts for inhibiting release of virus from an infected cell.

13. A method for inhibiting influenza virus replication in a subject, the method comprising administering to a subject having an influenza virus infection nafamostat mesylate and oseltamivir in effective amounts for inhibiting replication of the influenza virus.

14. A method for inhibiting influenza virus release from infected cells in a subject, the method comprising administering to a subject having an influenza virus infection nafamostat mesylate and oseltamivir in effective amounts for inhibiting release of the influenza virus from an infected cell.

15. The method of any one of the preceding numbered paragraphs, wherein the effective amount of nafamostat mesylate is 0.1 μM-100 μM at the drug delivery sites.

16. The method of any one of the preceding numbered paragraphs, wherein the effective amount of oseltamivir is 25 mg-100 mg at least once per day.

17. The method of any one of the preceding numbered paragraphs, wherein the effective amount reduces influenza virus titers by at least 2-fold.

18. The method of paragraph 17, wherein the effective amount reduces influenza virus titers by at least 10-fold.

19. The method of paragraph 17, wherein the effective amount reduces influenza titers by 2-fold to 20-fold.

20. The method of paragraph 17, wherein the effective amount reduces influenza titers by 2-fold to 100-fold.

21. The method of any one of the preceding numbered paragraphs, wherein the nafamostat mesylate and oseltamivir are administered within 24 hours-96 hours after influenza infection.

22. The method of paragraph 21, wherein the nafamostat mesylate and oseltamivir are administered within 72 hours after influenza infection.

23. The method of any one of the preceding numbered paragraphs, wherein the nafamostat mesylate and oseltamivir are administered prior to influenza virus infection.

24. The method of any one of the preceding numbered paragraphs, wherein the nafamostat mesylate and oseltamivir are administered simultaneously.

25. The method of any one of the preceding numbered paragraphs, wherein the nafamostat mesylate and oseltamivir are administered sequentially.

26. The method of paragraph 25, wherein the nafamostat mesylate is administered before the oseltamivir.

27. The method of any one of the preceding numbered paragraphs, wherein the nafamostat mesylate is administered in an effective amount to prevent emergence of influenza virus strains that are resistant to nafamostat mesylate.

28. The method of any one of the preceding numbered paragraphs, wherein the nafamostat mesylate is administered in an effective amount to reduce emergence of influenza virus strains that are resistant to nafamostat mesylate by at least 60% relative to a control.

29. A composition comprising nafamostat mesylate and oseltamivir.

30. The composition of paragraph 29 further comprising a pharmaceutically acceptable buffer.

31. A pharmaceutical composition comprising nafamostat mesylate and oseltamivir formulated in effective amounts to prevent and/or treat influenza virus infection in a subject.

32. The composition of any one of the preceding numbered paragraphs, wherein the composition comprises nafamostat mesylate and oseltamivir in effective amounts for inhibiting influenza virus replication.

33. The composition of any one of the preceding numbered paragraphs, wherein the effective amounts of nafamostat mesylate and oseltamivir are equivalent.

34. The composition of any one of the preceding numbered paragraphs, wherein the ratio of nafamostat mesylate to oseltamivir is 1:1-20:1.

35. The composition of any one of the preceding numbered paragraphs, wherein the ratio of nafamostat mesylate to oseltamivir is 1:1-1:20.

### EXAMPLES

### Example 1

### Combination Anti-Influenza Therapy Reduces Viral Titer

In this Example, an influenza infection model on a human airway chip (Benam, et al., 2016, Nature Methods, 13: 151-157) was used to test the efficacy of a new influenza virus therapy that combines the clinically-approved anticoagulant nafamostat mesylate and the clinically-approved anti-influenza virus drug oseltamivir (Tamiflu®) to effectively reduce viral titer in infected cells by more than 50%, in some instances, and to extend the therapy window by more than 24 hours.

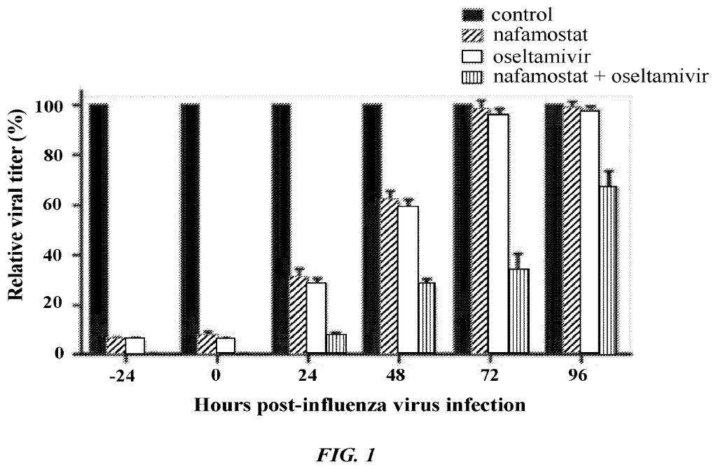

Human airway cells on the chip were infected with influenza virus and the therapeutic window for administering oseltamivir was detected. Oseltamivir was administered to infected cells at −24, 0, 24, 48, 72, and 96 hours (h) after influenza virus infection, and the resulting relative influenza virus titer was 5.4%, 5.4%, 28%, 58%, 96%, and 98%, respectively, compared to an untreated control (FIG. 1). This data shows that the therapeutic window for oseltamivir is approximately 48 hours post infection.

Similarly, nafamostat mesylate was administered to infected cells at −24, 0, 24, 48, 72, and 96 h after influenza virus infection, and the resulting relative influenza virus titer was 6.1%, 7.8%, 30.7%, 61%, 98%, and 98%, respectively, compared to an untreated control (FIG. 1). This data shows that the therapeutic window for nafamostat mesylate, similar to oseltamivir, is 48 hours post infection.

Oseltamivir targets influenza virus proteins (e.g., neuraminidase). Influenza virus may develop resistance to oseltamivir, rendering oseltamivir less effective at reducing influenza virus titer. Nafamostat mesylate inhibits serine proteases, which aid in influenza virus infectivity by Combination therapies which target both influenza virus proteins and human proteins that promote influenza virus infectivity are more effective at reducing influenza virus titer than individual therapies that target influenza virus or human proteins.

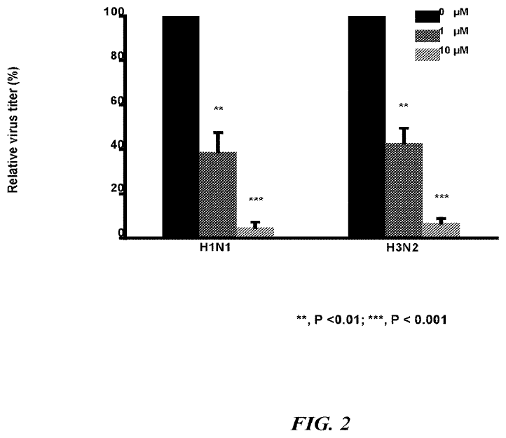

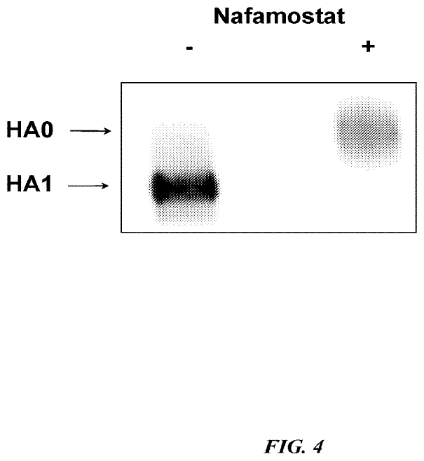

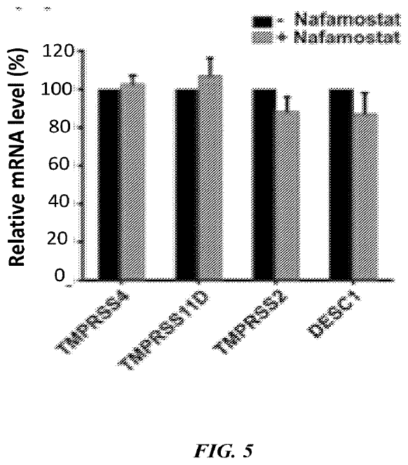

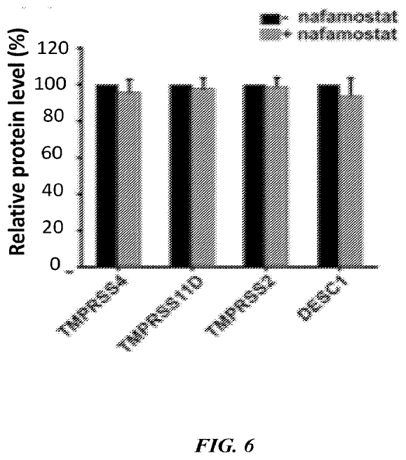

Nafamostat mesylate was found to exhibit inhibitory activity with a broad spectrum against influenza viruses, including H1N1 and H3N2 viruses (FIG. 2), when the influenza-infected airway was administrated by aerosol. When the effect of nafamostat mesylate on the cleavage of HA0 was tested, the data showed that nafamostat mesylate can efficiently block the cleavage of HA0 (FIG. 4). Further, nafamostat mesylate had no effect on the mRNA expression or protein expression of serine proteases (FIGS. 5, 6), indicating that nafamostat mesylate had no effect on the transcription or translation of these serine proteases. Collectively, these data show that nafamostat mesylate can block the HA0 cleavage by inhibiting the activity of host factors, e.g., serine proteases, and thus inhibit the influenza virus infection of the airway. Because the anti-influenza action mechanism of nafamostat mesylate is different from that of current anti-influenza drugs, which target virus proteins, the efficacy of the drug combination of nafamostat mesylate and oseltamivir was tested against influenza virus infection. Nafamostat mesylate was administered to infected human airway cells on the chip by aerosolization and oseltamivir was administered through the blood channel at −24, 0, 24, 48, 72, and 96 h after influenza virus infection. The resulting relative viral titer was 0.1%, 0.1%, 6.7%, 26.7%, 33.3%, and 66.7%, respectively, (FIG. 1). This data shows that the combination of nafamostat mesylate and oseltamivir exhibited synergistic anti-influenza efficacy and extended the therapeutic window from 48 hours to at least 96 hours.

The life cycle on influenza virus incudes three main stages: entry into host cell, replication in host cell, and release from host cell. Nafamostat mesylate inhibits the cleavage of the inactive hemagglutinin precursor HA0 into the active HA1 and HA2 hemagglutinin proteins, a step that is necessary for influenza virus entry into the host cells. Oseltamivir inhibits influenza virus release from the host cells. Inhibition of the influenza virus at two stages of its life cycle by the combination of nafamostat mesylate and oseltamivir leads to the increased efficacy and extends the therapeutic window for treating influenza virus infection (FIG. 1).

### Example 2

### Nafamostat Reduces Influenza Virus Resistance

Human patient-to-patient transmission of influenza virus was mimicked by passaging influenza virus from chip to chip under selection pressure of antiviral drugs to evaluate the propensity of anti-influenza drugs to induce viral resistance.

Human lung airway chips were infected with amantadine- and oseltamivir-sensitive influenza A/WSN/33 virus (H1N1) (MOI=0.01) and treated with 1 μM amantadine (Sigma-Aldrich), 1 μM oseltamivir acid, 10 μM nafamostat mesylate or left untreated for 48 hours. Amantadine or oseltamivir acid was perfused through the vascular channel of the human lung airway chip under flow (60 μl/h), while nafamostat mesylate was diluted in 20 μl PBS and delivered into the airway channel. The progeny viruses were isolated by incubating the airway channel with 50 μl PBS for 1 hour at 37° C., collecting the fluid, and then using it for infection in a new human lung airway chip. This procedure was repeated up to 30 times, and after each passage, progeny virus yields were quantified using the plaque formation assay. The virus yield of untreated human lung airway chips was set as 100%. When the progeny virus pool became resistant to drug treatment, the viruses were isolated through plaque purification and gene sequence to identify gene mutations.

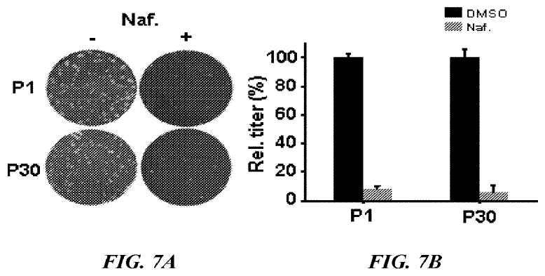

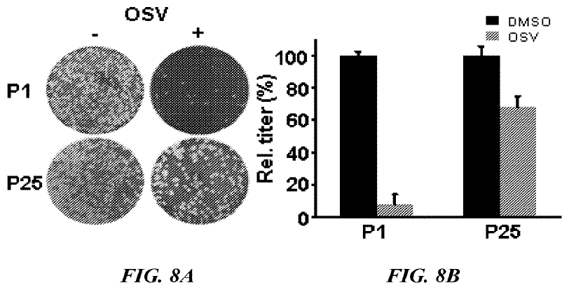

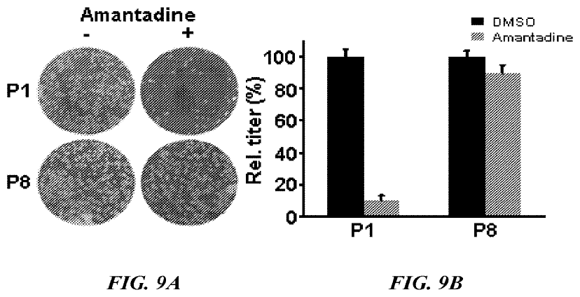

Surprisingly, influenza virus resistance to nafamostat mesylate was not detected, even when virus infection was transmitted over 30 human lung airway chip to human lung airway chip passages in the presence of nafamostat mesylate (FIGS. 7A-7B). Oseltamivir acid and amantadine induced the emergence of drug-resistant influenza viruses after 25 and 8 human lung airway chip to human lung airway chip passages, respectively (FIGS. 8A-8B, 9A-9B).

### Materials and Methods

Nafamostat mesylate (10 μM) was delivered by aerosolization into the airway channel of influenza-infected human airway on a chip cells at the indicated times. Forty-eight hours later, the virus samples were collected for detection of viral load. Oseltamivir acid (1 μM) was delivered under flow (60 μL/h) into the vascular channel of influenza-infected human airway on a chip cells at the indicated times. Forty-eight hours later, the virus samples were collected for detection of viral load.

Madin-Darby canine kidney II (MDCK.2) cells were seeded into 12-well plates to form a confluent monolayer. After the cells were washed with PBS, they were inoculated with 1 mL of 10-fold serial dilutions of influenza virus samples and incubated for 1 hour at 37° C. After unabsorbed virus was removed, the cell monolayers were overlayed with 1 mL of DMEM supplemented with 1.5% low melting point agarose and 2 μg/mL TPCK-treated trypsin. After incubation for 2-4 days at 37° C. in 5% CO₂, the cells were fixed with 4% paraformaldehyde and stained with crystal violet to visualize plaques. Virus titers were determined as plaque-forming units per milliliter.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. A method for reducing influenza virus titer in a subject, comprising administering to a subject infected with influenza virus a combination of nafamostat mesylate and oseltamivir in an effective amount to reduce influenza virus titer in the subject by at least 20% relative to a control.

2. The method of claim 1, wherein the combination is administered to the subject within 24 hours of infection in an effective amount to reduce influenza virus titer in the subject by at least 20% relative to a control, wherein the control is a subject administered only nafamostat mesylate or only oseltamivir within 24 hours of influenza virus infection.

3. The method of claim 1, wherein the combination is administered to the subject within 48 hours of infection in an effective amount to reduce influenza virus titer in the subject by at least 20% relative to a control, wherein the control is a subject administered only nafamostat mesylate or only oseltamivir within 48 hours of influenza virus infection.

4. The method of claim 1, wherein the combination is administered to the subject within 72 hours of infection in an effective amount to reduce influenza virus titer in the subject by at least 50% relative to a control, wherein the control is a subject administered only nafamostat mesylate or only oseltamivir within 72 hours of influenza virus infection.

5. The method of claim 1, wherein the combination is administered to the subject within 96 hours of infection in an effective amount to reduce influenza virus titer in the subject by at least 20% relative to a control, wherein the control is a subject administered only nafamostat mesylate or only oseltamivir within 72 hours of influenza virus infection.

6. The method of claim 1, wherein the nafamostat mesylate and the oseltamivir are administered simultaneously.

7. The method of claim 1, wherein the nafamostat mesylate and oseltamivir are administered sequentially.

8. The method of claim 7, wherein the nafamostat mesylate is administered before the oseltamivir is administered.

9. A method for preventing influenza virus infection in a subject, comprising administering to a subject a combination of nafamostat mesylate and oseltamivir in an effective amount to prevent influenza virus infection in the subject.

10. A method comprising contacting an airway cell with nafamostat mesylate and oseltamivir.

11. The method of claim 10, wherein the airway cell is contacted with the nafamostat mesylate and oseltamivir in effective amounts for inhibiting influenza virus replication.

12. The method of claim 10, wherein the airway cell is contacted with the nafamostat mesylate and oseltamivir in effective amounts for inhibiting release of virus from an infected cell.

13. A method for inhibiting influenza virus replication in a subject, the method comprising administering to a subject having an influenza virus infection nafamostat mesylate and oseltamivir in effective amounts for inhibiting replication of the influenza virus.

14. A method for inhibiting influenza virus release from infected cells in a subject, the method comprising administering to a subject having an influenza virus infection nafamostat mesylate and oseltamivir in effective amounts for inhibiting release of the influenza virus from an infected cell.

15. The method of claim 10, wherein the effective amount of nafamostat mesylate is 0.1 μM-100 μM at the drug delivery sites.

16. The method of claim 10, wherein the effective amount of oseltamivir is 25 mg-100 mg at least once per day.

17. The method of claim 10, wherein the effective amount reduces influenza virus titers by at least 2-fold.

18. The method of claim 17, wherein the effective amount reduces influenza virus titers by at least 10-fold.

19. The method of claim 17, wherein the effective amount reduces influenza titers by 2-fold to 20-fold.

20. The method of claim 17, wherein the effective amount reduces influenza titers by 2-fold to 100-fold.

21. The method of claim 10, wherein the nafamostat mesylate and oseltamivir are administered within 24 hours-96 hours after influenza infection.

22. The method of claim 21, wherein the nafamostat mesylate and oseltamivir are administered within 72 hours after influenza infection.

23. The method of claim 21, wherein the nafamostat mesylate and oseltamivir are administered prior to influenza virus infection.

24. The method of claim 10, wherein the nafamostat mesylate and oseltamivir are administered simultaneously.

25. The method of claim 10, wherein the nafamostat mesylate and oseltamivir are administered sequentially.

26. The method of claim 25, wherein the nafamostat mesylate is administered before the oseltamivir.

27. The method of claim 1, wherein the nafamostat mesylate is administered in an effective amount to prevent emergence of influenza virus strains that are resistant to nafamostat mesylate.

28. The method of claim 1, wherein the nafamostat mesylate is administered in an effective amount to reduce emergence of influenza virus strains that are resistant to nafamostat mesylate by at least 60% relative to a control.

29. A composition comprising nafamostat mesylate and oseltamivir.

30. The composition of claim 29 further comprising a pharmaceutically acceptable buffer.

31. A pharmaceutical composition comprising nafamostat mesylate and oseltamivir formulated in effective amounts to prevent and/or treat influenza virus infection in a subject.

32. The composition of claim 29, wherein the composition comprises nafamostat mesylate and oseltamivir in effective amounts for inhibiting influenza virus replication.

33. The composition of claim 29, wherein the effective amounts of nafamostat mesylate and oseltamivir are equivalent.

34. The composition of claim 29, wherein the ratio of nafamostat mesylate to oseltamivir is 1:1-20:1.

35. The composition of claim 29, wherein the ratio of nafamostat mesylate to oseltamivir is 1:1-1:20.
